(19) United States
(12) Patent Application Publication
(10) Pub. No.: US 2019/0142279 A1
(43) Pub. Date: May 16, 2019

(54) BODY TEMPERATURE MEASURING METHOD AND SYSTEM USING EARPHONE

(71) Applicant: WELT CORP., LTD., Seoul (KR)

(72) Inventors: Seong Ji Kang, Seoul (KR); Hye Kang Roh, Seoul (KR); Hwa Young Jeong, Incheon (KR)

(73) Assignee: WELT CORP., LTD., Seoul (KR)

(21) Appl. No.: 15/833,522

(22) Filed: Dec. 6, 2017

(30) Foreign Application Priority Data
Nov. 14, 2017 (KR) 10-2017-0151668

(51) Int. Cl. A61B 5/01; H04R 1/10; A61B 5/00; A61B 5/11

(52) U.S. Cl. CPC A61B 5/01; H04R 1/1016; H04R 1/1041; A61B 5/6817; A61B 2562/0271; A61B 5/0008; H04R 2460/07; A61B 2560/0223; A61B 5/1112

(57) ABSTRACT

Body temperature measuring method and system using earphone are provided, in which the body temperature measuring method includes connecting a user terminal to an earphone, measuring a first temperature at the earphone, measuring a second temperature at the earphone, and, at the user terminal, obtaining a body temperature by calibrating the second temperature by reflecting the first temperature. The first temperature is measured within a first predetermined time of connecting the user terminal to the earphone, and the second temperature is measured after a second predetermined time following connecting the user terminal to the earphone. The method may further include, when a GPS signal is received at the user terminal, receiving ambient temperature information of a position corresponding to the GPS signal, measuring a third temperature at the earphone, and obtaining a body temperature by calibrating the third temperature by reflecting the ambient temperature information.

### TECHNICAL FIELD

This application claims priority from Korean Patent Application No. 10-2017-0151668, filed on Nov. 14, 2017 in the Korean Intellectual Property Office, the disclosure of which is incorporated herein by reference in its entirety.

The present disclosure relates to a body temperature measuring method and system, and more particularly, to a body temperature measuring method and system using an earphone.

### BACKGROUND ART

In recent years, various services have been provided to support user healthcare using smartphones. For example, a variety of smartphone applications have been developed and commercialized, which may manage users' daily life habits such as exercise and eating habits, sleep patterns, biorhythms and so on. However, smartphone applications that have been commercialized so far are no better than just using the inertial sensor built in the smartphone to monitor the activities of the users, or receiving users' health information directly from the users.

It is important to measure and manage the biosignal such as body temperature to determine the health condition of the user. There is a need to develop a method for naturally measuring and managing biosignals using smartphones that users always carry around.

Recently, products with the body temperature measurement function using an earphone have been developed. However, there is a shortcoming that the accuracy is reduced since the ear temperature is affected by the ambient temperature and so on.

### SUMMARY

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

It is an object of the present disclosure to provide a body temperature measuring method and system, which are capable of accurately measuring a body temperature using an earphone by taking ambient temperature into consideration.

According to another aspect of the present disclosure, there is provided a biosignal measuring method using earphone, which may include connecting a user terminal to an earphone, measuring a first temperature at the earphone, measuring a second temperature at the earphone, and, at the user terminal, obtaining a body temperature by calibrating the second temperature by reflecting the first temperature.

The first temperature may be measured within a first predetermined time of connecting the user terminal to the earphone, and the second temperature may be measured after a second predetermined time following connecting the user terminal to the earphone.

The method may further include, when a GPS signal is received at the user terminal, receiving ambient temperature information of a position corresponding to the GPS signal, measuring a third temperature at the earphone, and obtaining a body temperature by calibrating the third temperature by reflecting the ambient temperature information.

The method may further include: when the GPS signal is received within the first time, providing ambient temperature information of a position corresponding to the GPS signal without measuring the first temperature, and obtaining a body temperature by calibrating the second temperature with the provided ambient temperature information.

A sensor driving signal may be transferred to the earphone to measure the second temperature during a mute interval in which an audio electric signal is not transferred from the user terminal to the earphone.

The sensor driving signal may be a signal having a frequency outside the audible frequency range.

According to an aspect of the present disclosure, there is provided a biosignal measuring system using earphone, which may include a user terminal configured to: cause an earphone to measure a first temperature and a second temperature; and obtain a body temperature by calibrating the second temperature by reflecting the first temperature.

According to the present disclosure, it is possible to naturally measure the user's body temperature and other biosignals while the user is wearing earphones on his or her ears and using a smartphone. In particular, the body temperature can be accurately measured by taking the ambient temperature into consideration.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a block diagram illustrating a configuration of a body temperature measuring system using an earphone according to an embodiment of the present disclosure;

FIG. 2 is a block diagram illustrating a detailed configuration of an earphone and a user terminal according to an embodiment of the present disclosure;

FIG. 3 is an audio waveform diagram provided to explain a mute interval according to the present disclosure;

FIG. 4 s a block diagram illustrating a detailed configuration of an earphone and a user terminal according to another embodiment of the present disclosure;

FIG. 5 is a view illustrating a right earbud of earphones according to an embodiment of the present disclosure;

FIG. 6 is a flowchart provided to explain an operation of a biosignal measuring system using an earphone according to an embodiment of the present disclosure; and

FIG. 7 is a flowchart provided to explain an operation of a biosignal measuring system using an earphone according to another embodiment of the present disclosure.

### DETAILED DESCRIPTION

Hereinafter, preferred embodiments of the present disclosure will be described in detail with reference to the accompanying drawings so that those with ordinary knowledge in the art can easily achieve the present disclosure.

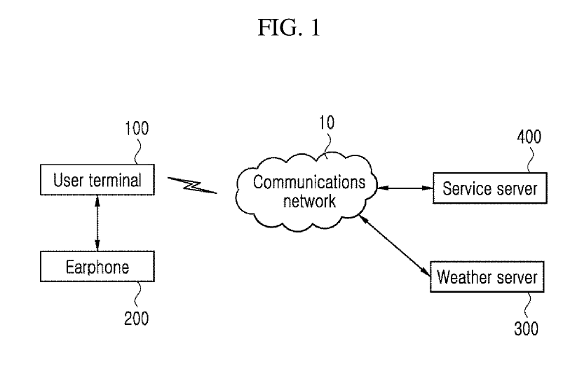

FIG. 1 is a block diagram illustrating a configuration of a body temperature measuring system using an earphone according to an embodiment of the present disclosure.

Referring to FIG. 1, a body temperature measuring system using an earphone according to an embodiment may include a user terminal 100, an earphone 200, a weather server 300, and a service server 400.

The user terminal 100, the weather server 300, and the service server 400 may exchange various kinds of information and data through a communication network 10.

The communication network 10 may include a variety of data communication networks including Local Area Network (LAN), Metropolitan Area Network (MAN), Wide Area Network (WAN), Internet, 3rd Generation (3G), 4th Generation (4G), Wi-Fi, Wireless Broadband Internet (WIBRO), or Long Term Evolution (LTE), whether wired or wireless, and using any communication method.

The user terminal 100 may be realized as an information communication terminal such as a smart phone, a tablet PC, a personal digital assistant (PDA), a web pad and so on, which is provided with a memory unit and also equipped with a microprocessor for computation capability.

The user terminal 100 may play content such as music, video and so on that includes sound information. The user terminal 100 may output the sound information contained in the content through a speaker (not shown) provided therein or may transfer the sound in the form of an audio electric signal to the earphone 200 so that the sound is outputted.

The user terminal 100 may measure the user's biosignal through the earphone 200. The user terminal 100 may receive the measured biosignal information from the earphone 200 and store the biosignal information in a memory (not shown). The user terminal 100 may display the biosignal information transferred from the earphone 200 on a screen.

The user terminal 100 may receive GPS signals. The user terminal 100 may receive the ambient temperature information of the current position corresponding to the received GPS signals from the weather server 300. The GPS signals are generally received outdoors, but not received indoors. That is, when the GPS signal is received, it may be determined that the user terminal 100 is currently positioned outdoors. Therefore, when the GPS signal is received, the ambient temperature information of the corresponding position provided from the weather server 300 may be determined as the ambient temperature at the current position of the user terminal 100.

The user terminal 100 may calibrate the body temperature measured at the earphone 200 using the ambient temperature information provided from the weather server 300. In addition, the user terminal 100 may first measure the ambient temperature of the place where the user is positioned by using the earphone 200, and then measure the body temperature of the user and calibrate the body temperature with the previously-measured ambient temperature. This will be described in detail below.

The biosignal measured at the earphone 200 may be a body temperature, an electrocardiogram (ECG), an electroencephalogram (EEG), an electromyogram (EMG), an electrooculogram (EOG), pulse rate and so on.

The weather server 300 may provide the ambient temperature information of the place corresponding to the current position of the user terminal according to a request of the user terminal 100.

The service server 400 may receive the measured biosignal information transferred from the user terminal 100 and store the same, or provide various services such as a health care service based on the biosignal information transferred from the user terminal 100.

According to the embodiment, the weather server 300 and the service server 400 may be operated by the same service provider or may be implemented by one device.

Hereinafter, an example in which the earphone 200 measures the body temperature will be described.

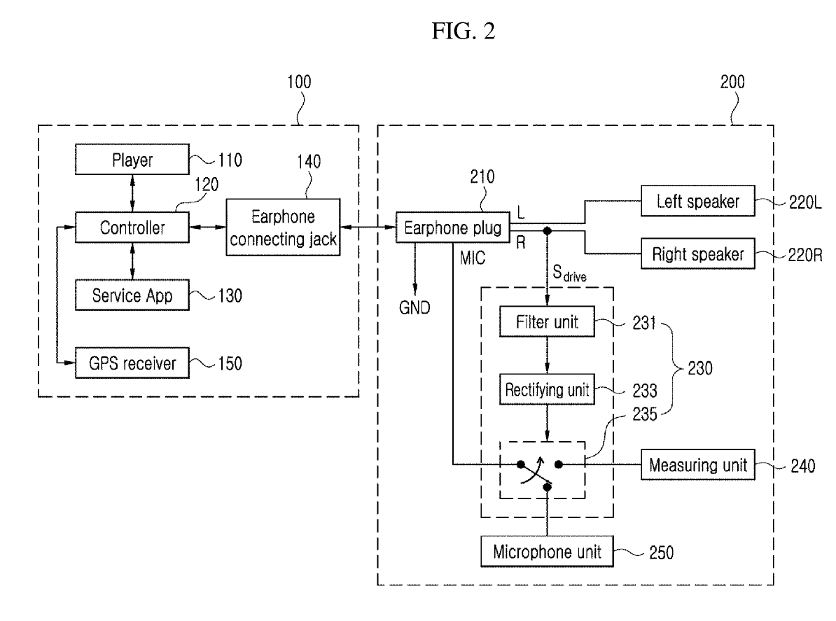

FIG. 2 is a block diagram illustrating a detailed configuration of an earphone and a user terminal according to an embodiment of the present disclosure.

Referring to FIG. 2, the user terminal 100 may include a player 110, a controller 120, a service application 130, an earphone connecting jack 140, a GPS receiver 150, and so on. Although not shown in FIG. 2, the user terminal 100 may also include a display module to visually output information, a speaker module to output information by sound, a memory module to store data and programs, a button or a touch panel to receive a user command, a communication module for communication with an external device, a battery module to supply operation power, an inertial sensor, a camera module and so on.

The player 110 performs a function of playing content including sound information such as music, video, etc., on the user terminal 100. The player 110 may be an audio playing application, a video playing application and so on.

The controller 120 may include a Central Processing Unit (CPU) and an operating system, and controls the overall operation of the user terminal 100. For example, Android smartphones may include CPU and Android platforms, and iPhone smartphones may include CPU and iOS.

The controller 120 may output an audio electric signal corresponding to the sound portion of the content played by the player 110 to the earphone 200 through the earphone connecting jack 140.

The controller 120 may monitor whether a mute interval occurs during playing of the content by the player 110, and may notify the service application 130 of it. Of course, the mute interval information may be directly transferred from the player 110 to the service application 130 instead of being transferred via the controller 120. The mute interval information may be information on the start or end of the mute interval.

When the player 110 continuously plays the songs included in the music playlist, there may generally be a mute interval between the songs in which no sound is outputted.

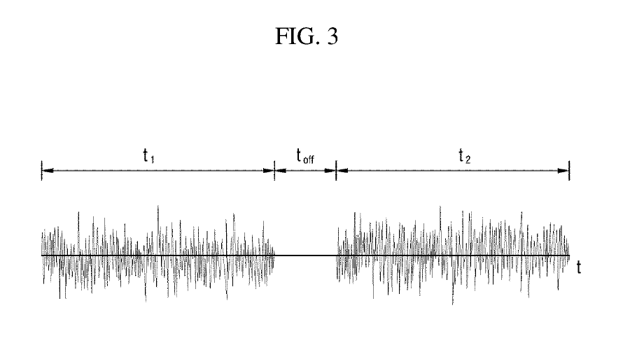

FIG. 3 is an audio waveform diagram provided to explain a mute interval according to the present disclosure.

As illustrated in FIG. 3, sound may be outputted while the first content is played for time t₁. And, there may be a mute interval in which no sound is outputted for a predetermined time t_{off}. Then sound may be outputted while the second content is played for time t₂.

Meanwhile, a mute interval may naturally occur between songs, but the mute interval may also be forcibly generated to measure a biosignal. For example, it is also possible to forcibly stop the operation of the player 110 from playing the content to generate a mute interval. Alternatively, while the player 110 is operated normally, in order to generate the mute interval, the output of the audio electric signal to the earphone 200 may be blocked. Accordingly, all the mute intervals may include the interval during which the audio electric signal is not transferred from the user terminal 100 to the earphone 200.

To measure the biosignal such as body temperature that can be measured in a relatively short time, the mute interval between songs may be utilized, while the mute interval may be forcibly generated for certain signals such as electrocardiogram that requires several tens of seconds or longer time for measurement.

Referring again to FIG. 2, the service application 130 may drive the measuring unit 240 provided in the earphone 200 to measure the body temperature. The service application 130 may receive the body temperature information measured at the earphone 200 to display the body temperature information on the screen of the user terminal 100 or store the body temperature information in a memory. The service application 130 may provide the body temperature information measured at the earphone 200 to the service server 400.

The service application 130 may be running in the background of the user terminal 100, measuring and collecting the body temperature at every predetermined time point through the earphone 200 without the user recognizing it. Of course, the body temperature measured at the earphone 200 may be received at a requested time point and displayed on the screen, in response to a request by a user for body temperature measurement that is made on a menu provided by the service application 130.

While the player 110 is continuously playing a plurality of songs according to the playlist, the service application 130 may transfer a sensor driving signal to the earphone 200 to cause the earphone 200 to measure the body temperature during the mute interval.

Meanwhile, the service application 130 may calibrate the body temperature measured at the earphone 200 by reflecting the ambient temperature of the place where the user is positioned. This will be described in detail below.

The earphone connecting jack 140 is connected to the earphone plug 210 to allow various signals to be exchanged between the user terminal 100 and the earphone 200.

The earphone connecting jack 140 may be realized as a 3.5 mm 4-pole terminal. Of course, depending on embodiments, the earphone connecting jack 140 may be a 2-pole terminal or a 3-pole terminal instead of a 4-pole terminal, or may have other types of terminals. Hereinafter, the embodiment of the present disclosure will be described with reference to an example of the 4-pole terminal.

The 4-pole terminal of the earphone connecting jack 140 may include a left audio signal terminal L, a right audio signal terminal R, a ground GND, and a microphone signal terminal MIC. The order of these terminals in the 4-pole terminal may vary between USA and European styles. The US style has an order of the left audio signal terminal L, the right audio signal terminal R, the ground GND, and the microphone signal terminal MIC. The European style has an order of the left audio signal terminal L, the right audio signal terminal R, the microphone signal terminal MIC, and the ground GND.

The user terminal 100 may transfer a left audio signal and a right audio signal to the earphone 200 through the left audio signal terminal L and the right audio signal terminal R. The user terminal 100 may receive the audio electric signal outputted from the microphone unit 250 from the earphone 200 through the microphone signal terminal MIC. Further, when there is a volume adjust button and so on in the earphone 200, a control signal corresponding to manipulation of the corresponding button may also be transferred.

The user terminal 100 may transfer a sensor driving signal S_{drive} through the left audio signal terminal L or the right audio signal terminal R. FIG. 1 illustrates an example in which the sensor driving signal S_{drive} is transferred through the right audio signal terminal R. The sensor driving signal S_{drive} is preferably a signal having a voltage of a predetermined intensity that has a frequency outside the human-audible frequency range. As a result, the sensor driving signal S_{drive} transferred to the left audio signal terminal L or the right audio signal terminal R may be prevented from being heard by the user through the left speaker 220L or the right speaker 220R. Specifically, the audio electric signal has a frequency corresponding to a frequency range of 20 Hz to 20,000 Hz, which is audible frequency range, and the sensor driving signal S_{drive} may be realized to have a frequency corresponding to a frequency band below 20 Hz or above 20,000 Hz. Of course, depending on the characteristics of the voice unit of the left speaker 220L or the right speaker 220R, the audio electric signal may be outputted as a sound in a narrower range than 20 Hz to 20,000 Hz. For example, the audio electric signal may be outputted as a sound only in a range of 40 Hz to 15,000 Hz. Thus, depending on embodiments, the frequency range of the audio electric signal and the frequency range of the sensor driving signal S_{drive} may vary.

Meanwhile, the sensor driving signal S_{drive} transferred to the left audio signal terminal L or the right audio signal terminal R at the vicinity of the boundary of the audible frequency band may have an effect such that it is heard by the user through the left speaker 220L or the right speaker 220R. In order to solve such problem, a filter (not shown) for filtering signals outside the audible frequency range (e.g., a band pass filter that passes only the signals in the audible frequency range) may be provided at the front end of the left speaker 220L or the right speaker 220R.

The earphone 200 may include an earphone plug 210, a left speaker 220L, a right speaker 220R, a switching unit 230, a measuring unit 240, and a microphone unit 250.

The earphone plug 210 has a structure corresponding to the earphone connecting jack 140. For example, when the earphone connecting jack 140 has an US version 4-pole terminal structure, the earphone plug 210 may also have an US version 4-pole terminal structure.

The first pole L of the earphone plug 210 may transfer the left audio electric signal transferred from the user terminal 100 to the left speaker 220L. The second pole R of the earphone plug 210 may transfer the right audio electric signal transferred from the user terminal 100 to the right speaker 220R. The third pole MIC of the earphone plug 210 may be selectively connected to one of the measuring unit 240 and the microphone unit 250 through the switching unit 230. In addition, the fourth pole GND of the earphone plug 210 is the ground.

The switching unit 230 is configured to selectively connect one of the measuring unit 240 or the microphone unit 250 to the third pole MIC.

The switching unit 230 may normally connect the microphone unit 250 and the third pole MIC, and then connect the third pole MIC and the measuring unit 240 when the sensor driving signal_{drive} is transferred from the user terminal 100.

When the sensor driving signal S_{drive} is inputted through the left audio signal terminal L or the right audio signal terminal R, the switching unit 230 may disconnect the third pole MIC from the microphone unit 250 to connect to the measurement unit 240. When the sensor driving signal is no longer applied, the switching unit 230 may re-connect the third pole MIC to the microphone unit 250.

The switching unit 230 may include a filter unit 231, a rectifying unit 233 and a switch 235 so as to process the sensor driving signal S_{drive} having a frequency outside the audible frequency range.

The filter unit 231 is configured to pass a sensor driving signal S_{drive} having a frequency outside the audible frequency range, while blocking the audio electric signal having a frequency corresponding to a frequency band of 20 Hz to 20,000 Hz (i.e., audible frequency range). To this end, the filter unit 231 may be realized as a high-pass filter that passes only a signal having a frequency above a predetermined frequency or a low-pass filter that passes only a signal having a frequency below a predetermined frequency. For example, the filter unit 231 may be realized as a high-pass filter that passes only signals above 20,000 Hz, or a low-pass filter that passes only signals below 20 Hz. That is, the filter unit 231 may be realized with the high-pass filter when the sensor driving signal S_{drive} has a frequency above the audible frequency range, while the filter unit 231 may be realized with the low-pass filter when the sensor driving signal S_{drive} has a frequency below the audible frequency range. With the filter unit 231, it is possible to prevent the audio electric signal from being applied to the switch 235 to cause malfunction.

The rectifying unit 233 is positioned between the filter unit 231 and the switch 235 to rectify the sensor driving signal S_{drive} that has passed through the filter unit 231 into direct current and output the direct current.

The switch 235 may be realized as a switching device that disconnects the third pole MIC from the microphone unit 250 to connect to the measuring unit 240 when a direct current voltage of a predetermined intensity or greater is inputted.

According to an embodiment, the switching unit 230 may also be realized such that, when the sensor driving signal is applied in the form of a pulse, the switching unit 230 connects the third pole MIC to the measuring unit 240 and automatically re-connects to the microphone unit 250 after a predetermined time has elapsed.

The circuit configuration of the switching unit 230 illustrated in FIG. 2 is merely an example. Another circuit configuration may be provided as long as it is able to connect the third pole MIC to the measuring unit 240 when the sensor driving signal S_{drive} is transferred.

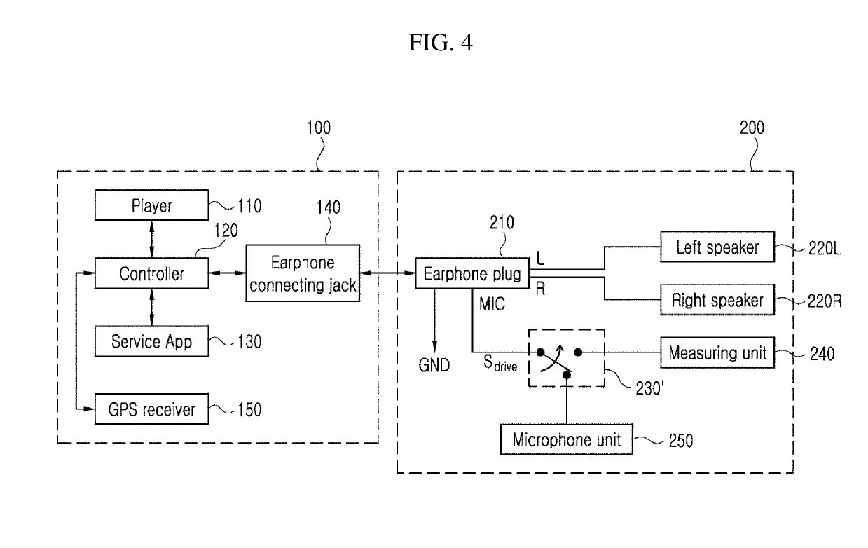

FIG. 4 is a block diagram illustrating a configuration of an earphone and a user terminal according to another embodiment of the present disclosure.

The embodiment illustrated in FIG. 4 differs from the embodiment illustrated in FIG. 1 in that a sensor driving signal S_{drive} is transferred from the user terminal to the switching unit 230′ through a microphone signal terminal MIC.

In the embodiment shown in FIG. 4, since the sensor driving signal S_{drive} is transferred through the microphone signal terminal MIC, the switching unit 230′ does not include the filter unit 231 and the rectifying unit 233, and may only include the switching device to disconnect the third pole MIC from the microphone unit 250 to connect to the measuring unit 240 when the input signal S is inputted.

Except for the switching unit 230′, the other components may operate in the same manner as the corresponding components having the same reference numerals in FIG. 2.

Meanwhile, according to an embodiment, unlike FIGS. 2 and 4, the switching unit may be realized as a toggle switch and so on. The user may selectively connect the third pole MIC to the microphone unit 250 or to the measuring unit 240 by manually operating the switching unit. The switching unit may be mounted on an earphone remote control (not shown) that may provide a volume control function and so on. When the user connects the measuring unit 240 to the third pole MIC by operating the toggle switch, the measuring unit 240 may measure the biosignal and transfer the measured biosignal to the user terminal 100 through the third pole MIC. The service application 130 may process the biosignal inputted through the third pole MIC to display on the screen or store in the memory.

The measuring unit 240 may be configured to measure a user's biosignal, and may include a sensor and a sensor circuit for this purpose. For example, when the biosignal of the measured subject is a body temperature, the measuring unit 240 may include a thermistor having electrical resistance value varying according to temperature. Of course, the measuring unit 240 may also use a sensor other than a thermistor to measure the body temperature.

The ear temperature may be affected by ambient temperature and so on. Accordingly, it is necessary to calibrate the body temperature measured at the earphone 200 by reflecting the ambient temperature. Meanwhile, when using a thermistor, a delay occurs in measuring temperature. For example, after a user inserts an earbud with a thermistor mounted therein into his or her ear, a delay occurs because it takes a certain amount of time before the resistance value of the thermistor changes to match the body temperature. That is, before the earbud is put into the ear, the resistance value of the thermistor is in agreement with the temperature of the environment, i.e., with the ambient temperature. The resistance value of the thermistor changes according to the body temperature only after a certain amount of time.

Using this characteristic of the thermistor, the service application 130 may first set the ambient temperature of the user environment based on the temperature measured at the measuring unit 240 within a predetermined time after the user terminal 100 and the earphone 200 are connected. The service application 130 may then determine user's body temperature based on the temperature measured at the measuring unit 240 after a predetermined time has elapsed, and may also calibrate the temperature by reflecting the ambient temperature.

The user terminal 100 may previously store in the memory the calibration data for calibrating the body temperature measured at the measuring unit 240 in accordance with the ambient temperature. That is, the ambient temperature of the environment around the user, the temperature actually measured at the measuring unit 240, and experimental data analyzing the correlativity between actual body temperature may be stored in advance in a memory in the form of a table or a graph. Alternatively, the user terminal 100 may be provided with, or refer to the calibration data from the outside through a communications network. The calibration data may be prepared in advance according to various criteria such as gender, age, season, time and so on. Of course, it is also possible to calibrate the body temperature measured at the measuring unit 240 through a predetermined algorithm.

Meanwhile, when the earbud has remained put in the user's ear for a certain period of time, the resistance value of the thermistor would have been changed according to the body temperature and accordingly, only the body temperature may be measured at the earphone 200, while the ambient temperature of the place where user is positioned may not be obtained anymore.

In order to solve such a problem, the user terminal 100 in outdoors where the GPS signal is received may receive the ambient temperature from the weather server 300 to use it to calibrate the body temperature measured at the earphone 200.

Meanwhile, the measuring unit 240 may include another sensor suitable for the biosignal of the measured subject. For example, when the biosignal of the measured subject is an electrocardiogram, an electroencephalogram, an electromyogram, an electrooculogram, a pulse rate, etc., the measuring unit 240 may additionally include another suitable sensor.

Further, while FIGS. 2 and 4 illustrate one measurement unit 240 by way of example, it is also possible to include a plurality of measuring units 240. The sensor driving signal may also be distinguished and transferred according to a sensor to be driven. For example, the body temperature sensor driving signal and the electrocardiograph sensor driving signal may be distinguished from each other.

Meanwhile, the measuring unit 240 may be provided on both the left earbud and the right earbud of the earphones 200, or may be provided on only one of the left and right earbuds in view of cost and so on.

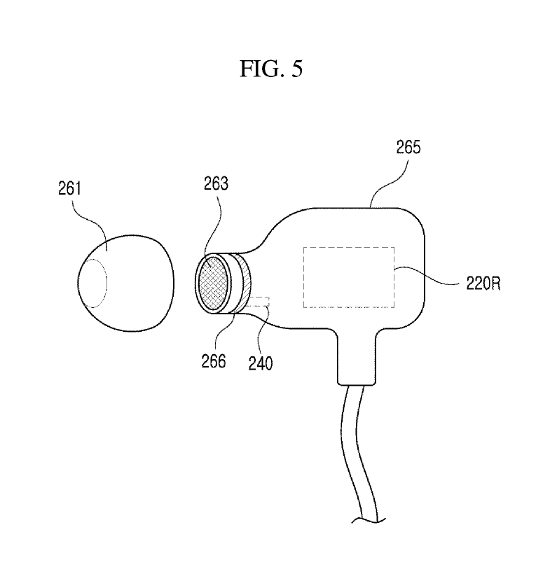

FIG. 5 is a view illustrating a right earbud of earphones according to an embodiment of the present disclosure.

Referring to FIG. 5, the right earbud may include an earpiece 261, a screen 263, a housing 265, a right speaker 220R, a measuring unit 240 and so on.

The housing 265 may have the right speaker 220R and the measuring unit 240 mounted therein. In addition, the sound outputted from the right speaker 220R may be outputted to the outside through the screen 263. In FIG. 4, the position where the right speaker 220R and the measuring unit 240 are mounted in the housing 265 is only an example, and the mounting position may vary according to embodiments.

The earpiece 261 may be mounted on one end of the housing 265 and inserted into the user's ear canal. The earpiece 261 may be made of an elastic material such as polysilicon, polyurethane, rubber, latex and so on so that it may be brought into close contact with the ear canal of a user when inserted. Particularly, the earpiece 261 may be made of a material such as polysilicon, polyurethane, rubber, latex and so on mixed or coated with a material having good thermal conductivity or electrical conductivity such that, when the earphone is inserted into the ear canal of the user, the biosignal is transferred to the measuring unit 240 efficiently.

A connecting member 266 may be formed on a portion of the housing 265. The connecting member 266 may be realized with a metal material or an elastic material having good thermal conductivity or electrical conductivity to electrically or thermally connect the earpiece 261 and the measuring unit 240. For example, as illustrated in FIG. 4, at least a portion of the connecting member 266 is exposed along the outer periphery of the housing 265 to contact the ear piece 261, while at least a portion of the connecting member 266 is exposed through the inner surface of the housing 265 to contact the measuring unit 240. As a result, the connecting member 266 may electrically or thermally connect the ear piece 261 mounted on one end of the housing 265 to the measuring unit 240 mounted inside the housing 265.

The earphone 200 may selectively include a microphone unit 250. For example, when the earphone 200 does not provide a microphone function, the earphone 200 may not include the microphone unit 250. Meanwhile, when the earphone 200 provides the volume control function, a volume control button (not shown) may be connected to the third pole MIC.

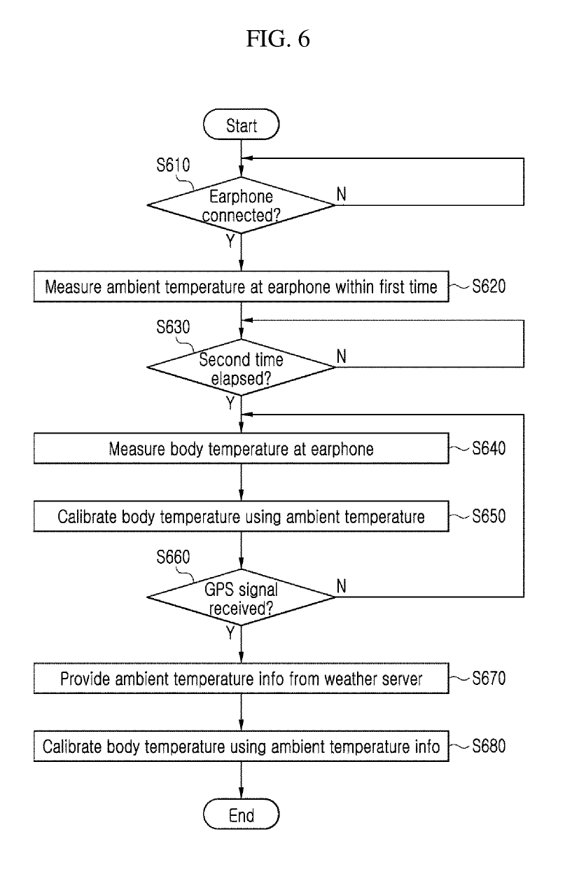

FIG. 6 is a flowchart provided to explain an operation of a biosignal measuring system using an earphone according to an embodiment of the present disclosure.

Referring to FIGS. 1 to 6, when the earphone 200 is first connected to the user terminal 100 at S610-Y, the service application 130 may transfer a sensor driving signal to the earphone 200 within a first predetermined time after the earphone 200 is connected to the user terminal 100 to cause the temperature measurement to be performed, at S620. For example, when the earphone 200 is connected to the user terminal 100, the temperature measurement may be immediately performed at the earphone 200. The temperature measured at S620 may be regarded as the ambient temperature. It is preferable that the first predetermined time is set as short as possible at S620 so that temperature measurement is performed at the earphone 200 before the resistance value of the thermistor changes according to the body temperature.

When a second predetermined time elapses after the earphone 200 is connected to the user terminal 100 at S630-Y, the service application 130 may transfer a sensor driving signal to the earphone 200 again to cause the temperature measurement to be performed at S640. Meanwhile, according to an embodiment, transferring the sensor driving signal at S640 may be performed only during the mute interval. The temperature measured at S640 may be regarded as a body temperature. The second predetermined time at S630 may be set to a degree necessary for the resistance value of the thermistor to be changed according to the body temperature.

In the above example, the first time may be set to be shorter than the second time.

Next, at S650, the service application 130 may obtain a calibrated body temperature by reflecting the temperature to the body temperature measured at S640.

Meanwhile, when the GPS signal is received at the user terminal 100 at S660-Y, the service application 130 may receive the ambient temperature information of the position corresponding to the GPS signal from the weather server 300 at S670.

Thereafter, the service application 130 may calibrate the body temperature measured at the earphone 200 using the ambient temperature information provided from the weather server 300, at S680.

Meanwhile, when GPS signal is not received at S660-N, the service application 130 may repeat the operations at S640 and S650, using the temperature measured at S620.

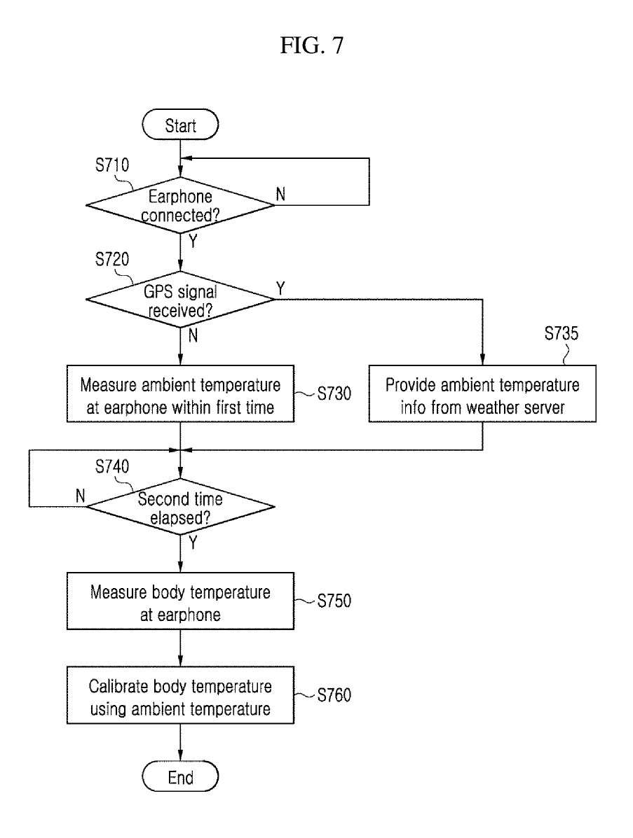

FIG. 7 is a flowchart provided to explain an operation of a biosignal measuring system using an earphone according to another embodiment of the present disclosure.

Referring to FIGS. 1 to 5 and 7, when the earphone 200 is connected to the user terminal 100 at 5710-Y, the service application 130 may determine whether a GPS signal is received at the user terminal 100 at S720.

When the GPS signal is not received at S720-N, the service application 130 transfers a sensor driving signal to the earphone 200 within a first predetermined time after the earphone 200 is connected to the user terminal 100 to cause the temperature measurement to be performed at S730.

When a second predetermined time elapses after the earphone 200 is connected to the user terminal 100 at S740-Y, the service application 130 may transfer a sensor driving signal to the earphone 200 again to cause the temperature measurement to be performed at S750.

Next, at S760, the service application 130 may obtain a calibrated body temperature by reflecting the ambient temperature to the body temperature measured at S750.

Meanwhile, when the GPS signal is received at the user terminal 100 at S720-Y, the service application 130 may receive the ambient temperature information of the position corresponding to the GPS signal from the weather server 300 at S735.

Thereafter, at S760, the service application 130 may calibrate the body temperature measured at the earphone 200 through the operations at S740 and S750 using the ambient temperature information.

Embodiments of the present disclosure include a computer-readable medium including program instructions for performing various computer-implemented operations. The medium records a program for executing the method described above. The medium may include program instructions, data files, data structures, etc., alone or in combination. Examples of such media include hardware devices, and so on configured to store and execute program instructions such as magnetic media such as hard disks, floppy disks and magnetic tape, optical recording media such as CD and DVD, floptical disk and magneto-optical media, ROM, RAM, flash memory, etc. Examples of the program instructions include machine language codes such as those generated by a compiler, as well as high-level language codes that may be executed by a computer using an interpreter, and so on.

The present disclosure has been described in detail. However, it should be understood that the detailed description and specific examples, while indicating preferred embodiments of the disclosure, are given by way of illustration only, since various changes and modifications within the scope of the disclosure will become apparent to those skilled in the art from this detailed description.

What is claimed is:

1. A biosignal measuring method using earphone, comprising:
connecting a user terminal to an earphone;
measuring a first temperature at the earphone;
measuring a second temperature at the earphone; and
at the user terminal, obtaining a body temperature by calibrating the second temperature by reflecting the first temperature.

2. The biosignal measuring method of claim 1, wherein the first temperature is measured within a first predetermined time of connecting the user terminal to the earphone, and
the second temperature is measured after a second predetermined time following connecting the user terminal to the earphone.

3. The biosignal measuring method of claim 1, further comprising:
when a GPS signal is received at the user terminal, receiving ambient temperature information of a position corresponding to the GPS signal;
measuring a third temperature at the earphone; and
obtaining a body temperature by calibrating the third temperature by reflecting the ambient temperature information.

4. The biosignal measuring method of claim 2, comprising:
when the GPS signal is received within the first time, providing ambient temperature information of a position corresponding to the GPS signal without measuring the first temperature, and obtaining a body temperature by calibrating the second temperature with the provided ambient temperature information.

5. The biosignal measuring method of claim 2, wherein a sensor driving signal is transferred to the earphone to measure the second temperature during a mute interval in which an audio electric signal is not transferred from the user terminal to the earphone.

6. The biosignal measuring method of claim 5, wherein the sensor driving signal has a frequency outside audible frequency range.

7. A biosignal measuring system using earphone, comprising a user terminal configured to cause an earphone to measure a first temperature and a second temperature and obtain a body temperature by calibrating the second temperature by reflecting the first temperature.

8. The biosignal measuring system of claim 7, wherein the first temperature is measured within a first predetermined time of connecting the user terminal to the earphone, and
the second temperature is measured after a second predetermined time following connecting the user terminal to the earphone.

9. The biosignal measuring system of claim 8, wherein, when a GPS signal is received, the user terminal is provided with ambient temperature information at a position corresponding to the GPS signal, causes the earphone to measure third temperature, and obtains a body temperature by calibrating the third temperature by reflecting the ambient temperature information.

10. The biosignal measuring system of claim 8, wherein, when the GPS signal is received within the first time, the user terminal is provided with ambient temperature information of a position corresponding to the GPS signal without measuring the first temperature, and obtains a body temperature by calibrating the second temperature with the provided ambient temperature information.

11. The biosignal measuring system of claim 8, wherein the user terminal transfers a sensor driving signal to the earphone to measure the second temperature during a mute interval in which an audio electric signal is not transferred from the user terminal to the earphone.

12. The biosignal measuring system of claim 11, wherein the sensor driving signal has a frequency outside audible frequency range.
